(19) United States
(12) Patent Application Publication
(10) Pub. No.: US 2026/0000810 A1
(43) Pub. Date: Jan. 1, 2026

(54) KAOLIN-BASED HEMOSTATIC MATERIAL WITH ANTI-INFLAMMATORY FUNCTION AND PREPARATION METHOD AND USE THEREOF

(71) Applicant: CHINA UNIVERSITY OF GEOSCIENCES (WUHAN), Wuhan (CN)

(72) Inventors: Huaming YANG, Wuhan (CN); Qianwen WU, Wuhan (CN); Hao WANG, Wuhan (CN)

(73) Assignee: CHINA UNIVERSITY OF GEOSCIENCES (WUHAN), Wuhan (CN)

(21) Appl. No.: 19/323,071

(22) Filed: Sep. 9, 2025

(30) Foreign Application Priority Data
Sep. 29, 2024 (CN) 202411366995.3

(51) Int. Cl. A61L 26/00

(52) U.S. Cl. CPC A61L 26/0004; A61L 2400/04

(57) ABSTRACT

The present disclosure relates to the technical field of hemostatic materials, and particularly relates to a kaolin-based hemostatic material with an anti-inflammatory function and a preparation method and use thereof. The kaolin-based hemostatic material with the anti-inflammatory function in the present disclosure includes CeO₂ nanoparticle-loaded kaolin. The preparation method includes: mixing kaolin, cerium chloride, sodium carbonate, and sodium chloride in a mass ratio, and ball-milling to produce a precursor; and roasting the precursor to produce the kaolin-based hemostatic material with the anti-inflammatory function. In the present disclosure, with low-cost and abundantly-available kaolinite and the conventional CeO₂ as raw materials, a CeO₂-loaded kaolin-based nanomaterial is prepared through mechanochemical synthesis. The prepared CeO₂/K nanomaterial has synergistic anti-inflammatory and hemostatic activities.

### CROSS-REFERENCE TO RELATED APPLICATIONS

This application claims priority to Chinese Patent Application No. 202411366995.3 with a filing date of Sep. 29, 2024. The content of the aforementioned application, including any intervening amendments thereto, is incorporated herein by reference.

### TECHNICAL FIELD

The present disclosure relates to the technical field of hemostatic materials, and particularly relates to a kaolin-based hemostatic material with an anti-inflammatory function and a preparation method and use thereof.

### BACKGROUND

In the treatment of diabetic foot, debridement is a necessary procedure to remove necrotic tissue including bone and abscess. The vasculopathy common in diabetic patients requires the diabetic patients to accept anticoagulation therapy to prevent cardiovascular events, which increases the risk of postoperative bleeding. Therefore, after debridement, not only an effective hemostatic measure is required, but also an inflammatory response needs to be controlled, so as to reduce the infection risk and promote the wound healing.

Currently, the sterile gauze remains the primary hemostatic material used in clinical practice. However, for diabetic patients undergoing long-term anticoagulant therapy, these conventional methods may be inadequate for effective bleeding control and may prolong the hospitalization time of patients. To improve the therapeutic effect for diabetic wounds, it is crucial to explore materials with superior hemostatic and anti-inflammatory properties.

Kaolin (K) is a natural mineral. Kaolin has shown great potential in the field of hemostasis due to excellent physicochemical properties and biocompatibility. Studies have shown that kaolin-based dressings exhibit a significant hemostatic effect for wounds and percutaneous vascular interventions. The patent application No. CN117462732A focuses on improving the hemostatic efficacy of kaolin and addressing the issues of easy agglomeration and pulverization. The invention patent No. CN109481731B adopts a nano-oxide to endow a kaolin-based hemostatic material with an antibacterial activity. As a result, the kaolin-based hemostatic material can play synergistic hemostatic and antimicrobial roles. The patent application No. CN116177587A discloses a mesoporous CeO₂ nanocomposite and confirms the strong oxidation resistance of the mesoporous CeO₂ nanocomposite. However, there is currently no kaolin-based material specifically designed for the synergistic hemostatic and anti-inflammatory effects required after the debridement of diabetic foot.

In addition, in the existing synthesis methods, toxic solvents or reagents are often adopted, such as NaOH and H₂SO₄. These substances may remain on the surface of nanoparticles to cause potential biological adverse reactions. The use of external stabilizers or end capping reagents may also compromise the biosafety of materials. Further, during the large-scale production, the consistency in sizes and shapes of nanoparticles can hardly be controlled, making it difficult to guarantee the purity and consistency for products.

### SUMMARY OF PRESENT INVENTION

An objective of the present disclosure is to provide a kaolin-based hemostatic material with an anti-inflammatory function and a preparation method and use thereof in view of the above-mentioned deficiencies in the prior art.

The present disclosure provides a kaolin-based hemostatic material with an anti-inflammatory function, including CeO₂ nanoparticle-loaded kaolin.

The present disclosure also provides a preparation method of the kaolin-based hemostatic material with the anti-inflammatory function, including: mixing kaolin, cerium chloride, sodium carbonate, and sodium chloride in a mass ratio, and ball-milling to produce a precursor; and roasting the precursor to produce the kaolin-based hemostatic material with the anti-inflammatory function.

Further, the mass ratio of the kaolin, the cerium chloride, the sodium carbonate, and the sodium chloride is 1:(0.2-2.35):1.25:1.15.

Further, the roasting is conducted at 200° C. to 800° C. for 0.5 h to 4 h.

Further, a heating rate for the roasting is 4° C./min to 6° C./min.

Further, the ball-milling is conducted for 0.5 h to 4 h at a rotational speed of 400 rpm to 560 rpm with a ball-to-material ratio of 130:(7.2-10.7).

Further, a container for the ball-milling is a 100 mL zirconia jar; media adopted for the ball-milling are zirconia balls with diameters of 10 mm, 8 mm, and 5 mm that are in a ratio of 2:3:5; and the ball-milling is conducted with a planetary ball mill according to a ball-milling strategy with a 30 min forward rotation and a 30 min reverse rotation in each cycle and a 5 min pause between every two cycles.

Further, before the ball-milling, the kaolin, the cerium chloride, the sodium carbonate, and the sodium chloride each are dried at 180° C. for 13 h.

Further, after being roasted, the precursor is subjected to centrifugal washing 2 times to 8 times, and then oven-dried at 45° C.

The present disclosure also provides a use of the kaolin-based hemostatic material with the anti-inflammatory function described above as a hemostatic material after debridement of diabetic foot.

In the present disclosure, with low-cost and abundantly-available kaolinite and the conventional CeO₂ as raw materials, a CeO₂-loaded kaolin-based nanomaterial is prepared through mechanochemical synthesis. The prepared CeO₂/K nanomaterial includes nanoparticles with high consistency in sizes and shapes, and has synergistic anti-inflammatory and hemostatic activities. In the present disclosure, the kaolinite provides a hemostatic effect and an effect of stabilizing CeO₂ nanoparticles. CeO₂ exhibits a catalytic activity to play an anti-inflammatory role. As a result, the present disclosure can achieve synergistic anti-inflammatory and hemostatic efficacy. Moreover, the present disclosure involves a short experimental cycle and a simple operating process, and can allow the green production and large-scale industrialization.

### BRIEF DESCRIPTION OF THE DRAWINGS

FIG. 1 is a schematic diagram of a synthetic process of CeO₂/K;

FIG. 2 shows X-ray diffraction patterns of Kaol and CeO₂/K;

FIG. 3 shows a scanning electron microscopy (SEM) image of Kaol;

FIG. 4 shows an SEM image of CeO₂/K;

FIG. 5 shows blood clotting indexes (BCIs) of CeO₂/K at 200° C. and 800° C.;

FIG. 6 shows BCIs of Kaol and CeO₂/K;

FIG. 7 shows in vitro nanozyme activities of Kaol and CeO₂/K; and

FIG. 8 shows a change in a tumor necrosis factor-α (TNF-α) level in lipopolysaccharide (LPS)-induced macrophages after a CeO₂/K action.

### DETAILED DESCRIPTION OF THE EMBODIMENTS

The technical solutions of the present disclosure are described in further detail below with reference to the specific embodiments and accompanying drawings, but the present disclosure is not limited thereto.

A preparation method of a kaolin-based hemostatic material with an anti-inflammatory function is as follows:

2.5 g of Na₂CO₃, 2.3 g of NaCl, 4.7 g of anhydrous CeCl₃, and 2 g of kaolinite are first weighed. The Na₂CO₃, the NaCl, and the anhydrous CeCl₃ each are dried overnight at a high temperature. Ball-milling is then conducted for a specified period of time in a planetary ball mill at a rotational speed of 560 rpm with a specified ball-to-material ratio to allow a mechanochemical action to produce a precursor for the kaolinite-based nanomaterial. The precursor is then placed in a muffle furnace and roasted at a specified temperature for a specified period of time. A roasting product is cleaned with deionized water under stirring, then dehydrated, and finally oven-dried at 45° C. for 24 h to produce a final product, which is the CeO₂-loaded kaolin-based nanomaterial (CeO₂/K).

### Example 1

In this example, CeO₂/K was prepared by the above method with different material ratios (Kaol:CeCl₃:Na₂CO₃:NaCl=1:(0.235-2.35):1.25:1.15).

2.5 g of Na₂CO₃, 2.3 g of NaCl, 0.47 g, 1.41 g, 2.342 g, 3.279 g, and 4.7 g of anhydrous CeCl₃, and 2 g of kaolinite were first weighed. The Na₂CO₃, NaCl, and anhydrous CeCl₃ each were dried overnight at a high temperature. The raw materials were ball-milled for 4 h in a planetary ball mill at a rotational speed of 560 rpm with a ball-to-material ratio of 130:10.7 to allow mechanochemical synthesis. A ball-milled material produced after the ball-milling was placed in a muffle furnace, heated at a heating rate of 5° C./min to 400° C., and then roasted at 400° C. for 1 h. A powder material produced after the roasting was transferred to a 50 mL centrifuge tube, subjected to centrifugal washing 6 times at 8,000 r/min, kept at 45° C. for 720 min, and dried to produce CeO₂/K of different material ratios.

### Example 2

In this example, CeO₂/K was prepared by the above method with different ball-milling times (0.5 h to 4 h).

2.5 g of Na₂CO₃, 2.3 g of NaCl, 4.7 g of anhydrous CeCl₃, and 2 g of kaolinite were first weighed. The Na₂CO₃, NaCl, and anhydrous CeCl₃ each were dried overnight at a high temperature. The raw materials were ball-milled for 0.5 h, 1 h, 2 h, 3 h, or 4 h in a planetary ball mill at a rotational speed of 560 rpm with a ball-to-material ratio of 130:10.7 to allow mechanochemical synthesis. A ball-milled material produced after the ball-milling was placed in a muffle furnace, heated at a heating rate of 5° C./min to 400° C., and then roasted at 400° C. for 1 h. A powder material produced after the roasting was transferred to a 50 mL centrifuge tube, subjected to centrifugal washing 6 times at 8,000 r/min, kept at 45° C. for 720 min, and dried to produce CeO₂/K.

### Example 3

In this example, CeO₂/K was prepared by the above method with different ball-to-material ratios (130:7.2, 130:8, 130:8.8, 130:10.7, and 130:12.6).

2.5 g of Na₂CO₃, 2.3 g of NaCl, 4.7 g of anhydrous CeCl₃, and 2 g of kaolinite were first weighed. The Na₂CO₃, NaCl, and anhydrous CeCl₃ each were dried overnight at a high temperature. The raw materials were ball-milled for 4 h in a planetary ball mill at a rotational speed of 560 rpm with a ball-to-material ratio of 130:7.2, 130:8, 130:8.8, 130:10.7, or 130:12.6. A ball-milled material produced after the ball-milling was placed in a muffle furnace, heated at a heating rate of 5° C./min to 400° C., and then roasted at 400° C. for 1 h. A powder produced after the roasting was transferred to a 50 mL centrifuge tube, subjected to centrifugal washing 6 times at 8,000 r/min, kept at 45° C. for 720 min, and dried to produce CeO₂/K.

### Example 4

In this example, CeO₂/K was prepared by the above method with different roasting temperatures (200° C. to 800° C.).

2.5 g of Na₂CO₃, 2.3 g of NaCl, 4.7 g of anhydrous CeCl₃, and 2 g of kaolinite were first weighed. The Na₂CO₃, NaCl, and anhydrous CeCl₃ each were dried overnight at a high temperature. The raw materials were ball-milled for 4 h in a planetary ball mill at a rotational speed of 560 rpm with a ball-to-material ratio of 130:10.7. A ball-milled material produced after the ball-milling was placed in a muffle furnace, heated at a heating rate of 5° C./min to 200° C., 300° C., 400° C., 500° C., or 800° C., and then roasted for 1 h at 200° C., 300° C., 400° C., 500° C., or 800° C. A powder produced after the roasting was transferred to a 50 mL centrifuge tube, subjected to centrifugal washing 6 times at 8,000 r/min, kept at 45° C. for 720 min, and dried to produce CeO₂/K.

### Example 5

In this example, CeO₂/K was prepared by the above method with different roasting times (0.5 h to 4 h).

2.5 g of Na₂CO₃, 2.3 g of NaCl, 4.7 g of anhydrous CeCl₃, and 2 g of kaolinite were first weighed. The Na₂CO₃, NaCl, and anhydrous CeCl₃ each were dried overnight at a high temperature. The raw materials were ball-milled for 4 h in a planetary ball mill at a rotational speed of 560 rpm with a ball-to-material ratio of 130:10.7. A ball-milled material produced after the ball-milling was placed in a muffle furnace, heated at a heating rate of 5° C./min to 400° C., and then roasted at 400° C. for 0.5 h, 1 h, 2 h, 3 h, or 4 h. A powder produced after the roasting was transferred to a 50 mL centrifuge tube, subjected to centrifugal washing 6 times at 8,000 r/min, kept at 45° C. for 720 min, and dried to produce CeO₂/K.

### Example 6

In this example, CeO₂/K was prepared by the above method with different numbers of centrifugal washing times (2 times, 4 times, 6 times, and 8 times) to remove the unsuccessfully-synthesized impurities.

2.5 g of Na₂CO₃, 2.3 g of NaCl, 4.7 g of anhydrous CeCl₃, and 2 g of kaolinite were first weighed. The Na₂CO₃, NaCl, and anhydrous CeCl₃ each were dried overnight at a high temperature. The raw materials were ball-milled for 4 h in a planetary ball mill at a rotational speed of 560 rpm with a ball-to-material ratio of 130:10.7. A ball-milled material produced after the ball-milling was placed in a muffle furnace, heated at a heating rate of 5° C./min to 400° C., and then roasted for 1 h at 400° C. A powder produced after the roasting was transferred to a 50 mL centrifuge tube, subjected to centrifugal washing 2 times, 4 times, 6 times, or 8 times at 8,000 r/min, kept at 45° C. for 720 min, and dried to produce CeO₂/K.

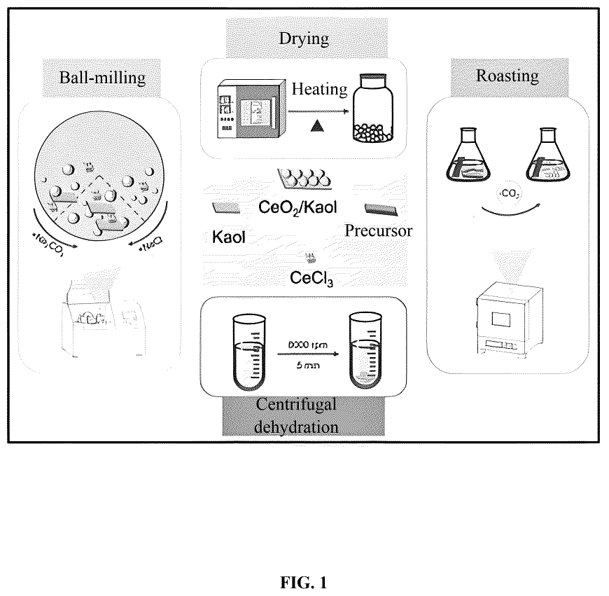

FIG. 1 is a schematic diagram of a synthetic process of CeO₂/K. It can be seen from this figure that the synthetic process of CeO₂/K is simple and eco-friendly.

The CeO₂/K prepared in Examples 1 to 6 was subjected to X-ray diffraction analysis. According to results, with either the increase of a ball-milling time or the decrease of a ball-to-material ratio, CeO₂ crystal grains are continuously refined. This is because the refinement of a powder in a ball-milling tank is accelerated with the increase of a ball-milling time and the decrease of a ball-to-material ratio to constantly reduce the crystal grains of a product. A change in the roasting temperature and the roasting time also causes a change in a crystal form of kaolinite. An increase in the roasting temperature and the roasting time improves the integrity of crystal grains of CeO₂, and increases the grain size. In addition, the number of dehydration times affects the residue of NaCl as a diluent. As the number of dehydration times increases, a characteristic absorption peak of NaCl is gradually reduced and completely disappears after 6 times of dehydration.

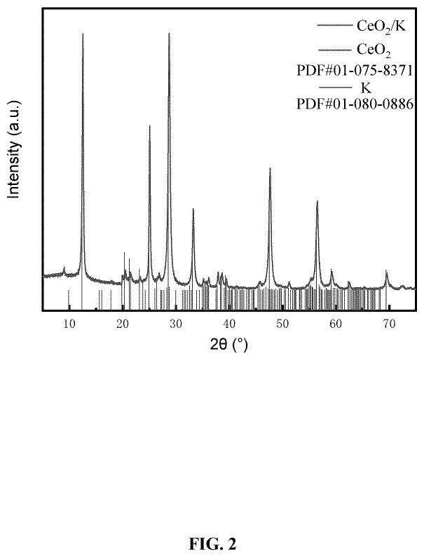

FIG. 2 shows an X-ray diffraction pattern of CeO₂/K synthesized after 6 times of dehydration in Example 6. As shown in FIG. 2, diffraction peaks observed at 2θ values of 28.55°, 33.08°, 47.48°, 56.34°, 59.08°, 69.41°, 76.70°, and 79.07° correspond to cubic fluorite-type CeO₂ with a spatial group Fm3m (PDF #01-075-8371), and diffraction peaks at 2θ values of 12.38°, 19.85°, 20.33°, 21.25°, 23.14°, and 24.90° correspond to Kaol (PDF #01-080-0886). It can be known that the synthesized product is a kaolinite/CeO₂ composite. In the CeO₂/K composite, a crystal lattice of kaolinite is not destructed, and kaolinite retains its original crystalline structure.

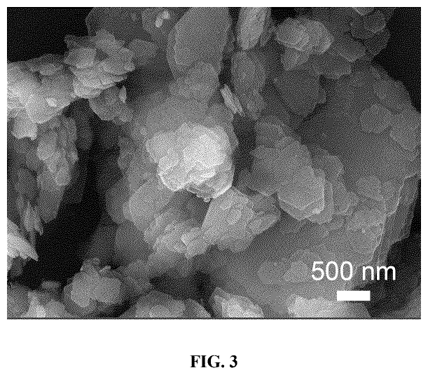

FIG. 3 shows an SEM image of Kaol. A microscopic morphology and structure of Kaol were analyzed by SEM. Kaol mainly presents a two-dimensional lamellar structure with a smooth surface and a distinct boundary.

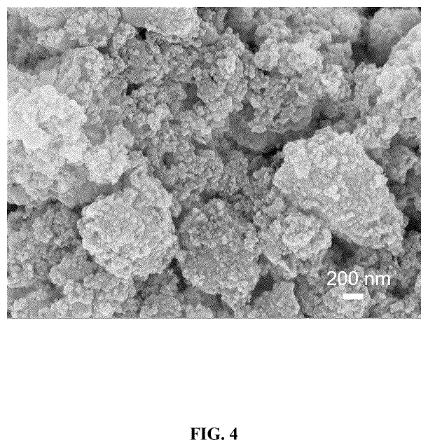

FIG. 4 shows an SEM image of CeO₂/K synthesized after 6 times of dehydration in Example 6. It can be clearly observed that CeO₂ is successfully anchored on the surface of kaolinite, CeO₂ particles are aggregates of small spherical particles, and kaolinite is completely covered by CeO₂ particles.

A coagulation test was carried out for the CeO₂/K prepared in Examples 1 to 6. It can be seen from results that, the longer the ball-milling time, the poorer the coagulation effect after kaolinite is transformed into an amorphous state. However, the ball-to-material ratio and the number of dehydration times have little influence on the coagulation effect. The roasting temperature and the roasting time also affect the coagulation effect of the material. When the roasting temperature is 800° C. and the roasting time is 1 h, the coagulation effect of the material significantly decreases. With the increase of the roasting temperature, the coagulation effect deteriorates. However, a too-low roasting temperature will affect the generation of CeO₂ particles. Therefore, the ball-milling time, roasting temperature, and roasting time can be comprehensively optimized by considering both the synergistic anti-inflammatory and hemostatic effects and the actual product requirements.

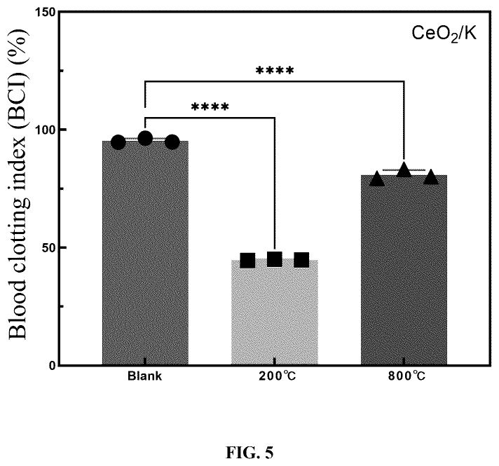

FIG. 5 shows BCIs of CeO₂/K materials prepared after roasting at 200° C. and 800° C. Effective hemostasis is the key to a hemostatic material. The lower the BCI, the stronger the hemostatic ability. In the coagulation test for CeO₂/K, BCI of the material produced at 200° C. decreases from 95.57±0.85% to 44.77±0.36%, and BCI of the material produced at 800° C. increases to 81.23±1.96%. These results confirm that the roasting temperature actually affects the coagulation effect of the material. The higher the roasting temperature, the higher the BCI. It indicates that a high temperature worsens the hemostatic performance of the material. Therefore, a reasonable roasting temperature should be selected.

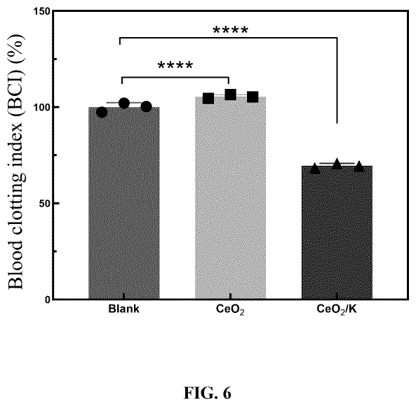

FIG. 6 shows BCIs of Kaol and CeO₂/K prepared after 6 times of dehydration in Example 5. In the coagulation test for CeO₂/K, the BCI decreases from 105.54±0.9% to 69.61±1.16%. It should be noted that CeO₂ cannot effectively stop bleeding, and even will worsen a hemostatic effect instead. However, the loading of CeO₂ with kaolinite can improve the hemostatic performance of the material.

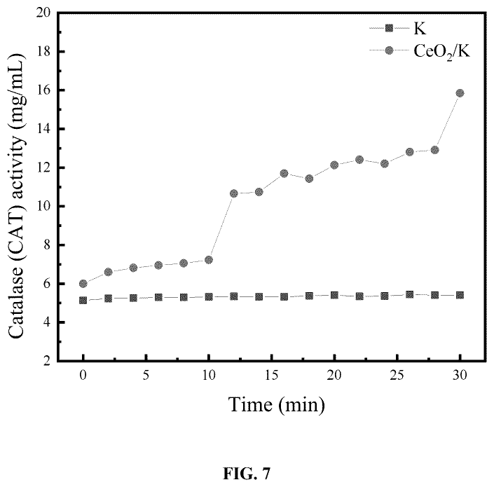

FIG. 7 shows in vitro nanozyme activities of Kaol and CeO₂/K prepared after 6 times of dehydration in Example 5. The clearance of reactive oxygen species (ROS) is a key factor in the suppression of inflammatory diseases by anti-inflammatory materials. The reduction of excessive ROS can prevent the further progression of diseases or promote the regeneration of damaged inflammatory tissues. Catalase (CAT) can scavenge superoxide anions and decompose hydrogen peroxide to produce molecular oxygen, thereby slowing down the occurrence of inflammation. According to the results of in vitro CAT nanoenzyme activities of Kaol and CeO₂/K, Kaol does not possess a CAT activity. However, after CeO₂ is loaded, the CAT activity increases, and the oxygen production capacity is further enhanced. It can be known that CeO₂/K has an in vitro CAT activity.

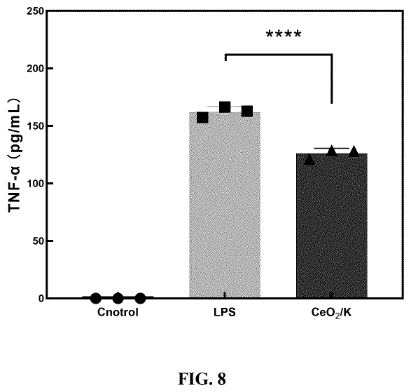

FIG. 8 shows a change in a TNF-a level in LPS-induced macrophages after a CeO₂/K action. In order to further understand an anti-inflammatory mechanism of CeO₂/K, a level of the related cytokine (TNF-α) in an inflammatory tissue was quantitatively analyzed by enzyme-linked immunosorbent assay (ELISA). According to results, CeO₂/K can effectively down-regulate the expression of the pro-inflammatory cytokine TNF-α to suppress the inflammatory death of macrophages in an inflammation-induced model, thereby protecting the cells from oxidative damage induced by H₂O₂.

What is not mentioned above can be acquired in the prior art.

Although some specific embodiments of the present disclosure have been described in detail by way of examples, those skilled in the art will appreciate that the above examples are provided for illustration only and not for limiting the scope of the present disclosure. A person skilled in the art can make various modifications or supplements to the specific embodiments described or replace them in a similar manner, but it may not depart from the direction of the present disclosure or the scope defined by the appended claims. Those skilled in the art should understand that any modification, equivalent replacement, and improvement made to the above embodiments according to the technical essence of the present disclosure shall be included in the protection scope of the present disclosure.

What is claimed is:

1. A preparation method of a kaolin-based hemostatic material with the anti-inflammatory function, comprising:
mixing kaolin, cerium chloride, sodium carbonate, and sodium chloride in a mass ratio, and ball-milling to produce a precursor; and
roasting the precursor to produce the kaolin-based hemostatic material with the anti-inflammatory function;
wherein the mass ratio of the kaolin, the cerium chloride, the sodium carbonate, and the sodium chloride is 1:(0.2-2.35):1.25:1.15;
wherein the roasting is conducted at 200° C. to 800° C. for 0.5 h to 4 h; and
wherein the ball-milling is conducted for 0.5 h to 4 h at a rotational speed of 400 rpm to 560 rpm with a ball-to-material ratio of 130:(7.2-10.7).

2. The preparation method according to claim 1, wherein a heating rate for the roasting is 4° C./min to 6° C./min.

3. The preparation method according to claim 2, wherein a container for the ball-milling is a 100 mL zirconia jar; media adopted for the ball-milling are zirconia balls with diameters of 10 mm, 8 mm, and 5 mm that are in a mass ratio of 2:3:5; and the ball-milling is conducted with a planetary ball mill according to a ball-milling strategy with a 30 min forward rotation and a 30 min reverse rotation in each cycle and a 5 min pause between every two cycles.

4. The preparation method according to claim 2, wherein before the ball-milling, the kaolin, the cerium chloride, the sodium carbonate, and the sodium chloride each are dried at 180° C. for 13 h.

5. The preparation method according to claim 2, wherein after being roasted, the precursor is subjected to centrifugal washing 2 times to 8 times, and then oven-dried at 45° C.

6. A kaolin-based hemostatic material with the anti-inflammatory function produced with the preparation method according to claim 1.
